(19) United States
(12) Patent Application Publication
(10) Pub. No.: US 2007/0239010 A1
(43) Pub. Date: Oct. 11, 2007

(54) Catheters with Laterally Deployable Elements and Linear Ultrasound Arrays

(75) Inventor: Jeremy A. Johnson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/279,265

(22) Filed: Apr. 11, 2006

(51) Int. Cl. A61B 8/00

(52) U.S. Cl. 600/439

(57) ABSTRACT

Catheters having laterally deployable elements (e.g., penetrators, needles, probes, electrodes, wires, etc.) and linear imaging arrays useable for imaging of such laterally deployable elements. These catheters are useable to perform or facilitate a wide variety of diagnostic and therapeutic functions, including but not limited to the delivery of drugs and other substances, accessing specific target locations within a subject's body, delivering diagnostic or therapeutic treatments to specific target locations within a subject's body, creating penetration tracts or passageways within a subject's body, etc.

### FIELD OF THE INVENTION

The present invention relates generally to methods and apparatus for medical treatment and more particularly to catheters having laterally deployable elements (e.g., penetrators, needles, probes, electrodes, wires, etc.) and linear imaging arrays useable for imaging of such laterally deployable elements.

### BACKGROUND

Numerous types of medical catheters have laterally deployable elements that may be advanced or extended laterally from the body of the catheter and used to perform some intended therapeutic or diagnostic function. Such laterally deployable elements include penetrators (e.g., straight or curved needles), probes (e.g., cryogenic probes, heating probes, etc.), wires (e.g., guidewires) and electrodes.

In many instances, it is desirable to image or visualize the laterally deployable element as it is advanced or extended from the catheter body. Moreover, the ability to image or visualize the laterally deployable element as well as nearby anatomical structures can be especially useful in situations where it is desired to advance or extend the laterally deployable element to a particular target anatomical structure and/or where is it desirable to avoid damaging or penetrating a particular anatomical structure.

The prior art has included a number of catheters that have laterally deployable elements along with on-board imaging apparatus useable to view the advancement or extension of the laterally deployable element. For example, catheters having laterally deployable tissue penetrating members (e.g., needles) in combination with imaging apparatus useable to visualize the deployment of the tissue penetrator and/or to provide a pre-indication of the trajectory on which the tissue penetrator will advance include those described in U.S. Pat. Nos. U.S. Pat. No. 5,830,222 (Makower), U.S. Pat. No. 6,068,638 (Makower), U.S. Pat. No. 6,159,225 (Makower), U.S. Pat. No. 6,190,353 (Makower, et al.), U.S. Pat. No. 6,283,951 (Flaherty, et al.), U.S. Pat. No. 6,283,983 (Makower, et al.), U.S. Pat. No. 6,375,615 (Flaherty, et al.), U.S. Pat. No. 6,508,824 (Flaherty, et al.), U.S. Pat. No. 6,544,230 (Flaherty, et al.), U.S. Pat. No. 6,579,311 (Makower), U.S. Pat. No. 6,602,241 (Makower, et al.), U.S. Pat. No. 6,655,386 (Makower, et al.), U.S. Pat. No. 6,660,024 (Flaherty, et al.), U.S. Pat. No. 6,685,648 (Flaherty, et al.), U.S. Pat. No. 6,709,444 (Makower), U.S. Pat. No. 6,726,677 (Flaherty, et al.) and U.S. Pat. No. 6,746,464 (Makower), the entire disclosure of each such United States patent being expressly incorporated herein by reference.

Also, U.S. Pat. No. 5,345,940 (Seward, et al.) describes a catheter that has an ultrasonic transducer proximate its distal end and a port from which a therapeutic device or the like may be deployed laterally from the catheter body under ultrasound visualization.

Additionally, U.S. Pat. No. 6,544,230 (Ishihra, et al.) describes a laser beam irradiation apparatus having a laterally deployable laser beam irradiation probe and an ultrasonic transmitter/receiver for emitting ultrasonic waves toward the region to which the laser beam is applied by the probe, receiving reflected waves from the irradiated region, converting the reflected waves into an electrical signal, a measuring device for measuring the temperature of the irradiated region in accordance with the electrical signal from the ultrasonic transmitter/receiver, and an output adjuster for adjusting the output of the laser generating device in accordance with a temperature signal from the measuring device.

There remains a need in the art for the development of new catheter deices having laterally deployable working elements and means for imaging the deployment of such working elements within the body of a human or animal subject.

### SUMMARY OF THE INVENTION

In accordance with the present invention there is provided a catheter device that comprises a catheter body, a laterally deployable working element that is advanceable or extendable from the catheter body and useable to perform a therapeutic or diagnostic function and a linear imaging array mounted longitudinally on or in the catheter body. The linear imaging array is useable to image the laterally deployable working element when it is advanced or extended from the catheter body. In some embodiments, the linear imaging array is also useable to image anatomical structures and may be used to locate an anatomical target site to which it is desired to advance or extend the working element. The linear imaging array may be any suitable type of imaging array, such as an ultrasound imaging array which displays an ultrasonic image on an extracorporeally located monitor or display screen. In some embodiments, indicia of the expected trajectory or path on which the working element will advance or extend may be superimposed or displayed on the image received from the linear imaging array. Additional imaging apparatus, such as a separate round imaging array may also be mounted on or in the catheter, along with the linear imaging array. The laterally deployable working element may comprise any suitable apparatus, device, energy form, composition or other element that is capable of performing or facilitating a diagnostic or therapeutic task, including but not limited to needles, curved needles, guidewires, catheters, cannulae, probes, cryogenic apparatus, cooling apparatus, heating apparatus, laser devices (e.g., laser wires), electrodes, electrosurgical probes, and antennae.

Further in accordance with the present invention, there is provided a method for performing a diagnostic or therapeutic function at a target site located outside of a body lumen. Such method generally includes the steps of (a) providing a catheter device that comprises i) a catheter body having a distal end, ii) a laterally deployable working element that is advanceable or extendable from the catheter body and useable to perform the therapeutic or diagnostic function and iii) a linear imaging array mounted longitudinally on or in the catheter body, said linear imaging array being useable to image the laterally deployable working element when it is advanced or extended from the catheter body; (b) positioning the catheter body within the body lumen; (c) advancing or extending the laterally deployable working element; (d) using the linear imaging array to image the working element while in its advanced or extended position and (e) using the working element to perform the diagnostic or therapeutic function at the target location. In some embodiments, the catheter device provided in Step (a) may be the same as that summarized hereabove in the immediately preceding paragraph.

Further aspects, details and embodiments of the present invention will be understood by those of skill in the art upon reading the following detailed description of the invention and the accompanying drawings.

### BRIEF DESCRIPTION OF THE DRAWINGS

FIG. 1 is a side view of one embodiment of a catheter device of the present invention having a laterally deployable element and a linear ultrasound array useable to visualize the advancement or extension of the laterally deployable element.

FIG. 2 is an enlarged, cut-away view of a distal portion of the catheter device of FIG. 1.

FIG. 3 is a rear perspective view of the catheter device of FIG. 1 with the laterally deployable element in an extended position and the linear ultrasound array being used to image the laterally deployable element as well as nearby anatomical structures.

FIG. 3A is distal end view of the showing of FIG. 3.

FIG. 3B is a right side view of the showing of FIG. 3.

The Figures are not necessarily to scale.

### DETAILED DESCRIPTION

The following detailed description, the accompanying drawings are intended to describe some, but not necessarily all, examples or embodiments of the invention. The contents of this detailed description and accompanying drawings do not limit the scope of the invention in any way.

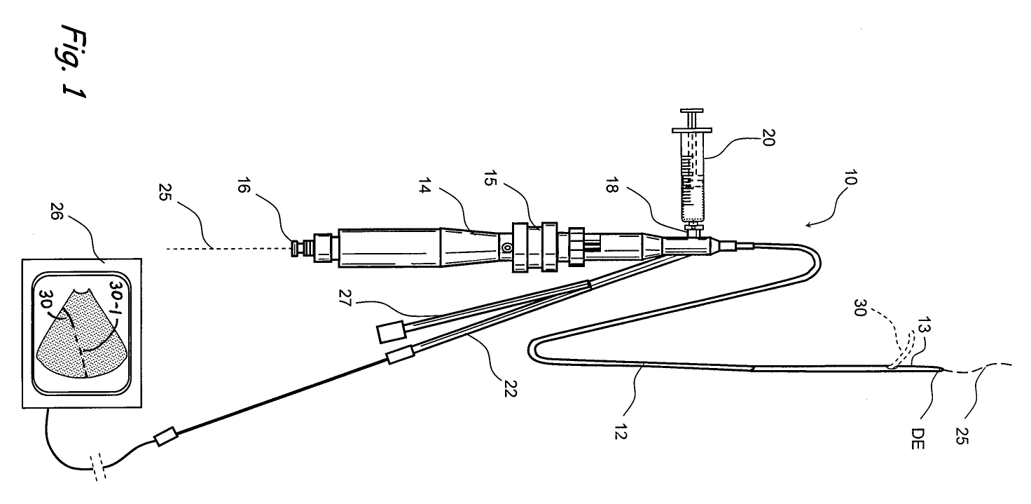

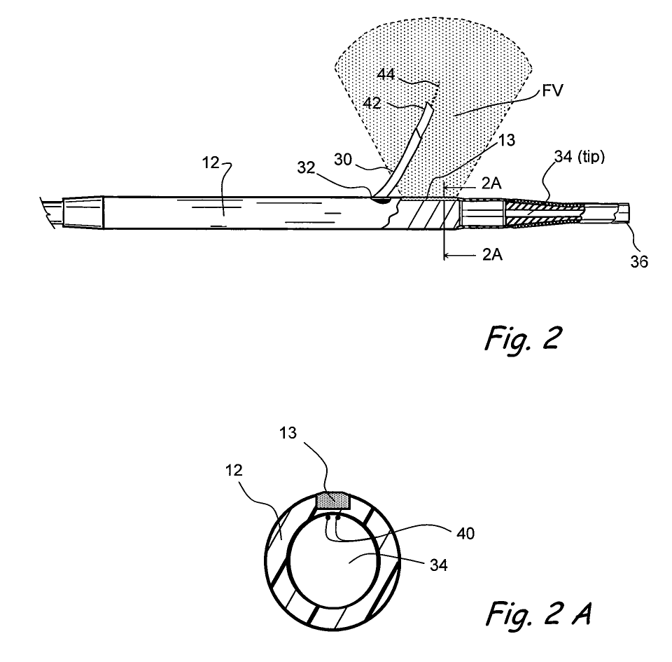

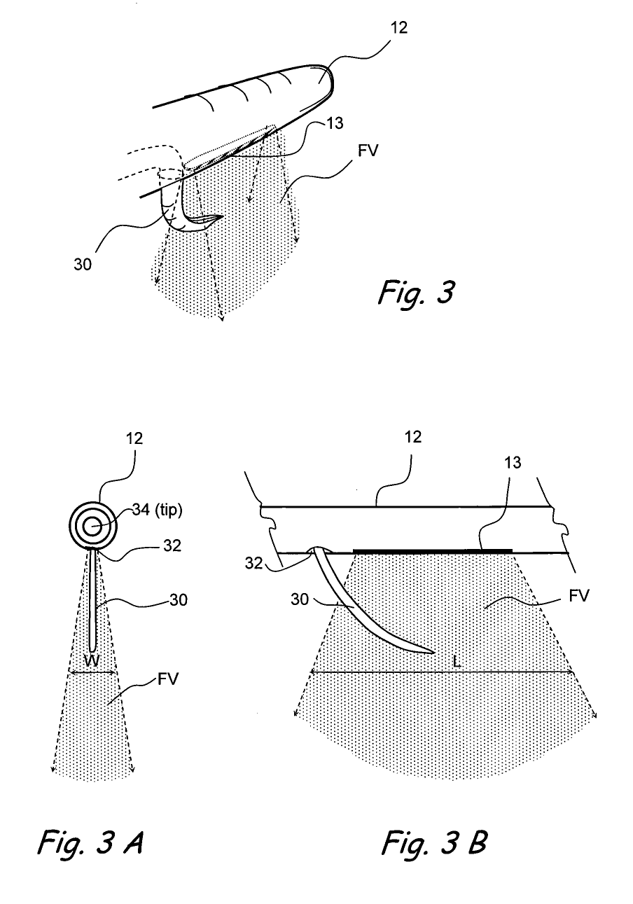

FIGS. 1-3B show one example of a catheter device 10 of the present invention. This catheter device 10 comprises an elongate catheter body 12 having a distal end DE, laterally deployable working element 30 that advances laterally out of side port 32 formed in catheter body 12 and a linear imaging array 13 that is useable to image the working element 30 and the surrounding area as the working element 30 advances from the catheter body 12. In the particular example depicted in the drawings, the laterally deployable working element comprises a hollow tissue penetrating member (e.g., a needle) having a lumen and an open distal tip. This tissue penetrating member may be formed of elastic or superelastic material (e.g., nickel-titanium alloy) and may be biased to a curved configuration, as shown. Those of skill in the art will appreciate that a substance, article or device may be delivered through the lumen of this hollow tissue penetrating member. For example, as seen in FIG. 2, additional working elements such as a delivery catheter 42 or guidewire 44 may be advanced through the lumen of this tissue penetrator. It should be understood that in the preferred embodiment delivery catheter 42 or guidewire 44, but not both at the same time, may be advanced through the lumen of the tissue penetrator, although in the figures both are depicted simply to illustrate that either may be advanced. It should also be appreciated, however, that the laterally deployable working element 30 need not necessarily comprise a tissue penetrating member as seen in this example, but may additionally or alternatively comprise any other apparatus(es) or device(s) capable of performing diagnostic or therapeutic functions, including but not limited to guidewires, catheters, cannulae, probes, cryogenic apparatus, cooling apparatus, heating apparatus, laser devices (e.g., laser wires), electrodes, electrosurgical probes, and antennae.

A handpiece 14 is provided on the proximal end of the catheter body 12, as shown in FIG. 1. The laterally deployable working element 30 is longitudinally moveable between a retracted position where it is substantially retracted within the catheter body 12 and an extended position wherein it has been longitudinally advanced out of side port 32 such that it extends on a trajectory or path away from the catheter body 12. The handpiece 14 comprises an advancement/retraction knob 15 which may be pushed in the distal direction to advance the working element 30 from its retracted position to its extended position and pulled in the proximal direction to retract the working element 30 from its extended position to its retracted position. An adjustable stop member 17 limits the extent of distal advancement of the advancement/retraction knob 15, thereby controlling the length from the side port 32 to the distal tip of the working element 30 when the working element 30 is fully extended. It is to be appreciated, however, that longitudinal advancement and retraction of the working element 30 is only an example of the way in which the working element 30 may laterally deploy from the catheter body. Various other modes of advancement or extension of the working element 30 may be employed, for example the working element may telescope, fold or pivot instead of longitudinally advancing and retracting.

In the particular embodiment shown in the drawings, a first proximal side arm 27 is connected to the proximal end of the lumen of the tissue penetrating member such that a substance, article or device may be delivered through the proximal side arm 27 and through that lumen. In the showing of FIG. 2, a guidewire 44 and a delivery catheter have been introduced through side arm 27 and advanced through the penetrator lumen such that they extend out of an beyond the distal end of the penetrator. In the preferred embodiment, the guidewire used is any suitable commercially available guidewire while the delivery catheter used may be the IntraLume™ Microcatheter (Medtronic, Inc. Minneapolis, Minn., USA) is used.

A tapered distal tip member 36 having a lumen 38(tip) is mounted on the distal end of the catheter body 12. A through lumen 34 extends from a port 16 on the handpiece 14, through the catheter body 12 and is continuous with the distal tip lumen 34(tip) such that a continuous through lumen 38, 38(tip) extends from proximal port 16 though the open distal end of distal tip member 36. A guidewire may pass through this lumen 38, 38 (tip) for over-the-wire advancement of the catheter device 10. It will be appreciated that, in some alternative embodiments, the lumen 38 may terminate proximally in a side opening in the catheter body 12, thereby providing a rapid exchange type guidewire lumen. Also in the embodiment shown, an infusion port 18 is optionally formed on the handpiece 14 in communication with lumen 38 such that an infusion apparatus 20 (e.g., a syringe, intravenous tube, pump, injector, etc.) may be used to infuse fluid (e.g., saline solution, radiographic contrast medium, etc.) through lumen 38, 38(tip) and out of the open distal end of the tip member 46. A valve (e.g., a Tuohy-Borst valve) may be provided on proximal port 16 to secure a guidewire when desired and/or to form a fluid tight seat at proximal port 16 when fluid is being infused through infusion port 18.

The linear imaging array 13 may be mounted in or on the catheter body 12 in such a way as to provide an image from a limited field of view FV. This field of view FV may extend from one side of the catheter body and may encompass a substantial portion of the trajectory or path on which the working element 30 will advance or extend. Additionally, in at least some embodiments, the field of view FV may extend to a distance that is sufficient to image the intended target location to which the working element 30 is to be advanced or extended. In some embodiments, the field of view FV may constitute a defined radial area that extends from the catheter body in the same lateral direction in which the working element 30 will advance or extend. In such embodiments, the operator may rotate and longitudinally move the catheter body 12 within a body lumen (e.g., a blood vessel lumen) until the intended target site is located within (or centered within) the field of view FV as seen on a monitor which displays the image received by the linear imaging array 13. This will allow the operator to use the image obtained from the linear imaging array to adjust the longitudinal position and/or rotational orientation of the catheter body 12 relating the intended target location prior to actual advancement or extension of the working element to ensure, or to at least increase the likelihood, that subsequent advancement or extension of the working element 30 will cause the working element 30 to enter the intended target site rather than some other location.

Also, in some embodiments, an indicator of the expected trajectory or path on which the working element 30 will advance may be superimposed or otherwise shown on the image displayed by the imaging system 26 such that the operator may then adjust the longitudinal position and/or rotational orientation of the catheter body 12 until the indicator of the expected trajectory or path on which the working element 30 is within the target site seen on the image. For example, the expected trajectory (depicted as 30-1 in FIG. 1) may be pre-programmed into the imaging system and, once the catheter is recognized by the system, said the expected trajectory would be superimposed on the display. This will ensure, or least increase the likelihood, that subsequent advancement or extension of the working element 30 will cause the working element 30 to enter the intended target site rather than some other location.

In the example of FIGS. 1-2A, connector wire(s) 40 extend from the linear imaging array 13, through catheter body 12, through proximal side arm 22 and are connected to an imaging system 26 which displays the image received by the linear imaging array 13. However, in some other embodiments, the connector wires 40 may be replaced by wireless technology known in the art for sending and receiving signals between the linear imaging array 13 and the extracorporeally located imaging/display system 26.

The linear imaging array may comprise a linear ultrasound array consisting of a plurality of ultrasound emitters/receivers disposed in a substantially straight line and operating at a common frequency. For example, the linear array may consist of between 2 to 128 ultrasound emitters/receivers disposed in a substantially linear fashion along the catheter. The ultrasound emitters/receivers preferably operate at between 5 to 50 MHz. linear.

It is to be appreciated that this catheter device 10 may be used for many different purposes wherein it is desired to advance or extend any laterally deployable working element 30 from a catheter to a desired location. In some instances, the target location may be within in the wall of a vessel in which the catheter body 12 is positioned. For example, the wall of a blood vessel is made up of several layers (e.g., tunica intima, tunica media and tunica adventitia or outer coat) and the working element 30 may be advanced from the lumen of the blood vessel to a location within the blood vessel wall (e.g., into the adventitia or outer coat of the blood vessel. This technique may facilitate advancement of a tubular working element 30 into the vessel wall such that a desired diagnostic or therapeutic substance can be injected directly into the vessel wall. For example, this technique may be used for injection of drugs into an artery wall to deter restenosis of the artery following an angioplasty procedure.

In other instances, the target location may be outside of the wall of the luminal anatomical structure in which the catheter body 12 is positioned and the working element 30 may be advanced or extended all the way through the luminal wall to the desired target site. For example, the catheter body 12 may be positioned within the lumen of one blood vessel and a working element 30 comprising a tissue penetrator may be advanced to a target location within the lumen of another blood vessel. Such vessel-to-vessel penetration may be utilized to create, or to facilitate the creation of, a passageway or fistula between two blood vessels. Or, as a further example, the target location may be a natural or man made cavity or structure located adjacent to or a spaced distance away from the body lumen in which the catheter body 12 is positioned. Examples of such possible target locations include organs, tumors, body cavities, previously implanted devices such as substance reservoirs or drug eluting devices, etc. Examples of possible target locations, ancillary procedures and applications of the present invention include but are not limited to those described in U.S. Pat. Nos. U.S. Pat. No. 5,830,222 (Makower), U.S. Pat. No. 6,068,638 (Makower), U.S. Pat. No. 6,159,225 (Makower), U.S. Pat. No. 6,190,353 (Makower, et al.), U.S. Pat. No. 6,283,951 (Flaherty, et al.), U.S. Pat. No. 6,283,983 (Makower, et al.), U.S. Pat. No. 6,375,615 (Flaherty, et al.), U.S. Pat. No. 6,508,824 (Flaherty, et al.), U.S. Pat. No. 6,544,230 (Flaherty, et al.), U.S. Pat. No. 6,579,311 (Makower), U.S. Pat. No. 6,602,241 (Makower, et al.), U.S. Pat. No. 6,655,386 (Makower, et al.), U.S. Pat. No. 6,660,024 (Flaherty, et al.), U.S. Pat. No. 6,685,648 (Flaherty, et al.), U.S. Pat. No. 6,709,444 (Makower), U.S. Pat. No. 6,726,677 (Flaherty, et al.) and U.S. Pat. No. 6,746,464 (Makower).

Also, in some instances, the catheter 12 may be inserted into a man made passage such as a guidewire tract or neo-lumen created in the wall of a blood vessel past an occlusive lesion (e.g., a chronic total occlusion) and a working element 30 comprising a penetrating member then be used to penetrate from the catheter body back into the true lumen of that blood vessel, at a location downstream of the obstruction. In such procedures, if the penetrating member has a lumen 32, a guidewire may be advanced through the penetrator lumen and into the true lumen of the blood vessel downstream of the obstruction. Thereafter, the working element 30 may be retracted and the catheter device 10 removed, leaving the guidewire in place. A stent may then be delivered over that guidewire and used to stent the man made guidewire tract (e.g., “neo-lumen) thereby providing a bypass conduit for blood flow around the obstructive lesion.

In applications of the invention where a flowable substance is to be delivered, the working element 30 may comprise a needle, catheter or other substance delivery device(s) that can advance or extend into a target site and used to deliver the desired substance to the target site. It will be appreciated that in some such embodiments, the working element 30 may comprise a hollow penetrator (e.g., a needle) having a lumen. The substance may in some cases be delivered directly though the lumen of that penetrator or, in other cases, a separate delivery catheter 42 may be advanced through the lumen of the penetrator as seen in FIG. 2 and the substance may then be delivered through that delivery catheter 42. In some of these cases, the delivery catheter 42 may be capable of penetrating through tissue and the working element 30 comprising a penetrator may be advanced to a first location some distance form the target site. The delivery catheter 42 may be advanced through the working element 30 and through any intervening tissue, from the first location to the target site. Examples of the types of substances that may be delivered include but are not limited to: contrast agents or other agents that provide an enhanced image of the target site, traceable substances that may be used to determine the rate at which the substance distributes away from or is otherwise inactivated at the target site or other pharmacokinetic or biodistributive parameters or variables, drugs, proteins, cells (e.g., stem cells, nerve cells, progenator cells, myoblasts, myocytes, secretory cells, pancreatic islet cells, dopamine secreting cells, endothelial cells, hepatocytes, cloned cells, cells grown in cell culture, genetically modified cells, and combinations thereof), angiogenic substances (e.g., vascular endothelial growth factor (VEGF), fibroblast growth factors (FGF), epidermal growth factor (EGF), platelet-derived growth factor (PDGF), hepatocyte growth factor (HGF)or scatter factor, heparin combined with an adenosine receptor agonist, nerve cell growth factor (NGF), and combinations thereof), other agents that increase vascularity of an ischemic target site, myogenic substances, neurogenic substances, genes, gene therapy compositions, genetic material in combination vectors (e.g., viruses), stem cells of a type that will mature in situ into a type of cell that is currently deficient, substances that promote the growth of myocytes in tissue that is necrotic or characterized by a lack of living myocytes, secretory cells that secrete a substance (e.g., dopamine, insulin, a particular neurotransmitter) that is deficient, step F comprises insulin secreting cells, glial cell line-derived neurotropic factor (GDNF), nerve growth factor, neuro-immunophilin ligand, poly ADP-Ribose polymerase, and combinations thereof.

In applications of the invention where an article is to be delivered to the target site, it will be appreciated that in some cases such article may be introduced directly through a lumen formed in the working element 30 or the desired article may be otherwise delivered though, over or released from the working element. Examples of the types of articles that may be delivered using catheter devices 10 of the present invention include but are not limited to; bulking agents, substance eluting implants, radioactive implants, embolic members, markers, and radiopaque markers.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A catheter device comprising:
a catheter body having a distal end;
a laterally deployable working element that is advanceable or extendable from the catheter body and useable to perform a therapeutic or diagnostic function; and
a linear imaging array mounted longitudinally on or in the catheter body, said linear imaging array being useable to image the laterally deployable working element when it is advanced or extended from the catheter body.

2. A device according to claim 1 wherein the linear imaging array comprises an ultrasound array useable to provide an ultrasonic image.

3. A device according to claim 1 wherein the catheter body further comprises a port from which the laterally deployable working element advances or extends and wherein the linear imaging array is positioned adjacent to said port.

4. A device according to any of claims 1, 2 or 3 wherein the linear imaging array is useable, prior to advancement or extension of the laterally deployable element, to provide an image of anatomical structures that lie in the path on which the laterally deployable element will travel if subsequently advanced or extended while the catheter body remains in its current position.

5. A device according to any of claims 1, 2, 3 or 4 wherein the laterally deployable working element comprises a member that is advanceable or extendable from the catheter body.

6. A device according to claim 5 wherein said member comprises a tissue penetrating member.

7. A device according to claim 6 wherein said tissue penetrating member comprises a needle.

8. A device according to claim 6 wherein said tissue penetrating member comprises a curved needle.

9. A device according to claim 6 wherein said tissue penetrating member has a lumen through which a substance, article or device may be delivered.

10. A system comprising a device according to claim 9 in combination with a substance, article or device that is deliverable through or over of the tissue penetrating member.

11. A system according to claim 10 wherein the linear imaging array is also useable to provide an image of the substance, article or device after it has been delivered through the lumen of the tissue penetrating member.

12. A device according to claim 9 wherein the working element further comprises a delivery catheter that is advanceable though the lumen of the tissue penetrating member.

13. A device according to claim 12 wherein the tissue penetrating member has a distal end and wherein the delivery catheter penetrates further through tissue as it advances beyond the distal end of the tissue penetrating member.

14. A device according to claim 12 or 13 wherein the linear imaging array is also useable to provide an image of the delivery catheter.

15. A system comprising a catheter device according to claim 12 or 13 in combination with a substance, article or device that is deliverable through the delivery catheter after the delivery catheter has been advanced through the tissue penetrating member.

16. A system according to claim 15 wherein the linear imaging array is also useable to provide an image of the substance, article or device after it has been delivered through the delivery catheter.

17. A device according to claim 1 further comprising:
further comprising an image display system that is adapted to display, prior to advancement or extension of the working element, an image obtained from the linear imaging array and to superimpose on said image an indication of the path or trajectory on which the working element is expected to subsequently advance or extend if actually advanced or extended while the catheter body is in its current position.

18. A device according to claim 1 further comprising:
a circular imaging array positioned on or in the catheter body.
